(19) United States
(12) Patent Application Publication
(10) Pub. No.: US 2006/0263275 A1
(43) Pub. Date: Nov. 23, 2006

(54) Cabinet and sterilizing lamp

(75) Inventor: Ernst Lobach, Eschen (LI)

Correspondence Address: BROWDY AND NEIMARK, P.L.L.C.;624 NINTH STREET, NW, SUITE 300, WASHINGTON, DC, 20001-5303, US

(73) Assignee: Liconic AG, Mauren (LI)

(21) Appl. No.: 11/430,194

(22) Filed: May 9, 2006

(30) Foreign Application Priority Data
May 9, 2005 (CH) 0814/05

(51) Int. Cl. B01J 19/08

(52) U.S. Cl. 422/186

(57) ABSTRACT

The chamber of a cabinet, e.g. a climate controlled cabinet, is provided for a controlled storage or preparation of biological samples or goods. A high pressure mercury lamp in the chamber allows to efficiently generate UV-radiation, ozone and heat and to sterilize the chamber.

### RELATED APPLICATION

The present application claims the priority of Swiss patent application 00814/05 filed May 9, 2005, the disclosure of which is incorporated by reference herein in its entirety.

### BACKGROUND OF THE INVENTION

The invention relates to a cabinet having a chamber for the controlled storage or preparation of biological samples or goods, such as a climate controlled cabinet. The invention also relates to a method for sterilizing the interior of such a cabinet as well as to a high-pressure mercury lamp.

Clinical sterilization technology teaches various methods for killing of germs, in particular the sterilization by means of hot air or gases, such as ethylene oxide or formaldehyde. Generally, the clinical technologies are effective against a limited range of germs only.

A typical climate controlled cabinet has a chamber for receiving biological samples or goods and can be used as incubator or freezer, e.g in biological laboratories. Before introducing new samples, it has to be reset to a defined initial state; in particular the chamber including stationary mechanical structures therein must be as free as possible from germs of any kind.

### SUMMARY OF THE INVENTION

Hence, it is an object of the invention to provide a cabinet and method of this type that allows an efficient, wide range sterilization.

This object is achieved by a cabinet comprising

a chamber for a controlled storage or preparation of biological samples or goods and

at least one high-pressure mercury lamp located to sterilize said chamber prior to receiving said goods.

In a further aspect of the invention, the above object is met by a method for sterilizing a chamber of a cabinet for storing biological samples or goods comprising the step of sending UV-light from a high-pressure mercury lamp into said chamber while, at the same time, heating said chamber with heat from said high-pressure mercury lamp and generating ozone in said chamber with said UV-light.

In another aspect, the invention relates to a a method for sterilizing a chamber of a cabinet for storing biological samples or goods comprising the steps of

cycling air in said chamber and, simultaneously,

sending UV-light from a high-pressure or low-pressure mercury lamp into said chamber, thereby generating a level of ozone lethal for germs.

In yet a further aspect, the invention relates to a high-pressure or low-pressure mercury lamp comprising at least one optical filter having a better optical transmission for radiation between 180 nm and 230 nm than for radiation between 230 nm and 280 nm.

The invention is based on the concept of using several different techniques simultaneously in order to eradicate a wide range of germs. It exploits the fact that, during a decontamination phase, there are no biological probes or goods in the chamber. Hence, it is possible to use non-conventional methods, namely the gassing by ozone as well as irradiation with hard UV-light (wavelengths from 200 nm)

The invention uses a combination of three (per-se known) measures for germ eradication, namely:
UV-irradiation
ozone gassing
hot air

The sterilizing effect of UV-light is described by J. Kiefer in “Ultraviolette Strahlen”, Walter de Gruyter, Berlin 1977. The sterilizing effect of ozone is described by M. Horvatz, L. Blitzky and J. Hüttner in “Ozone”, Elsevier, Amsterdam 1985. Hot air sterilization is generally known.

The invention relies on a single device generating all three sterilizing effects in simple manner in order to reduce costs of manufacturing and ownership.

This single device is a high-pressure mercury lamp. In this type of lamps, the major part of the light is generated directly or indirectly from the radiation of the mercury at a partial pressure above 100 kilopascal. A possible standardized type of high-pressure mercury lamps is described by European standard EN 60188.

High-pressure mercury lamps and their electronic drivers have been known for a long time (see e.g. W. Elenbaas, “Quicksilberdampf-Hochdrucklampen”, Philips Technische Bibliothek, Eindhoven 1966) . However, such lamps have, to the best of our knowledge, so far not been used for sterilization in the context of the present invention.

In contrast to this, low-pressure mercury lamps have been used for a long time for sterilizing objects. For the present application, however, low-pressure mercury lamps are generally too week in view of light intensity and ozone generation, unless they are used over an extended period of time in a closed chamber, in particular if the air is cycled therein.

DE 102 032 34 describes a method for decontaminating a flow box, wherein ozone is generated by an ozone generator outside the flow box and then led into the flow box. A UV-lamp is mentioned as one possible type of ozone generator (see claim 2 of that application). However, an operation based on this method has various disadvantages:
On the one hand, part of the generated ozone decays immediately upon contact with the walls, in particular with the walls of the duct leading from the ozone generator to the flow box. This effect becomes particularly distinct when leading the ozone through a filter since filters have large surfaces.
On the other hand, there is no visual contact between the UV-lamp and the working space of the flow box for which reason the germ sterilizing effect of the UV-light cannot be used directly,

In an advantageous embodiment, the present invention does not suffer from these disadvantages because the mercury lamp is either directly placed in the chamber or it is positioned to emit light into that chamber, in particular light below 250 nm or 230 nm. It may be permanently installed in the chamber of be inserted therein temporarily when a decontamination is required.

### BRIEF DESCRIPTION OF THE FIGURES

The invention will be better understood and objects other than those set forth above will become apparent when consideration is given to the following detailed description thereof. Such description makes reference to the annexed drawings, wherein

FIG. 1 is a sectional view of an advantageous high-pressure mercury lamp,

FIG. 2 is a schematic view of a climate controlled cabinet with a high-pressure mercury lamp,

FIG. 3 is a sectional view long line S through the high-pressure mercury lamp of FIG. 1 in horizontal mounting position,

FIG. 4 is a horizontal sectional view of a climate controlled cabinet with rotating carousel, transport unit and horizontal high-pressure mercury lamp, and

FIG. 5 is a horizontal view of a second climate controlled cabinet with two storage racks, transport unit and horizontal high-pressure mercury lamp.

### DETAILED DESCRIPTION OF THE INVENTION

In the following, we first describe the design of an advantageous embodiment of the high-pressure mercury lamp, and then its arrangement in a climate controlled cabinet.

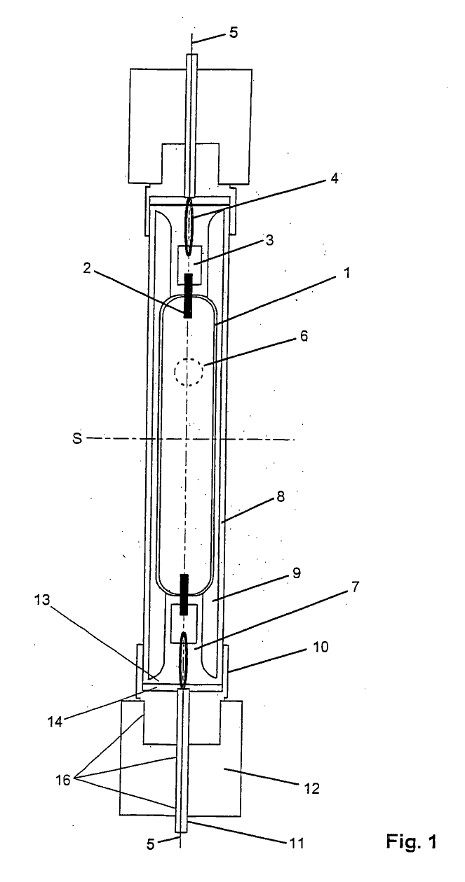

The high-pressure mercury lamp of FIG. 1 has a cylindrical arc tube 1 of quartz glass, which is closed at both ends. At each end, a tungsten electrode 2 projects into arc tube 1. The lamp is symmetric to a symmetry mirror plane S extending perpendicularly to its longitudinal axis 5.

The electrodes 2 are connected to incoming electrical conductors 5 by means of vacuum tight lead throughs 3 and wires 4. The electrical conductors 5 are connected to a power supply 34 of the high-pressure mercury lamp. The insulation requirements are not described in detail since they are known to the person skilled in the art.

Arc tube 1 is filled with a gas filling 6, as it is typical for high-pressure mercury lamps. It is held by two rod members 7 of glass or quartz glass, each extending between a collar 13 and arc tube 1. An outer tube 8 is fused in gas tight manner to the rod members 7 via the collars 13. A gap 9 between outer tube 8 and arc tube 1 is filled with an oxygen-free gas, in particular nitrogen, for increasing the thermal conductance between arc tube 1 and outer tube 8.

All parts 1, 7, 8 and 13 consist advantageously of synthetic quartz glass for obtaining a high UV-transmission since it is in particular the short wave UV region between 180 and 230 nm that contributes to ozonogenesis. Accordingly, light with a wavelength smaller 250 nm, in particular smaller than 230 nm, should be sent into the chamber to be sterilized.

Housing arc tube 1 within outer tube 8 has two primary functions. Namely, outer tube 8 increases ozone generation and it can act as a container for any mercury escaping from arc tube 1.

When the high-pressure mercury lamp is burning, the wall of arc tube 1 reaches, at the height of plane S, temperatures of up to 1200K. However, above 800 KV, ozone starts to decompose to a substantial degree such that, without outer tube 8, ozone formed close to the lamp would decay quickly. On the other hand, the outer wall of outer tube 8 reaches a maximum temperature of 700 K only.

A further increase of the ozone yield of the lamp of FIG. 1 can be achieved by using a blocking filter substantially -blocking radiation between 230 nm and 280 nm. The book by M. Horvath, L. Bilitzky, J. Hüttner, “Ozone”, Elsevier, Amsterdam 1985 teaches on page 21, FIG. 10 that light in this wavelength range is absorbed by ozone and leads to a decomposition of ozone generated with light of shorter wavelengths.

If a high ozone yield is desired (at the cost of a lower UV light yield), this can be achieved by using a filter blocking UV light between 230 nm and 280 nm while simultaneously transmitting light below 230 nm, in particular between 180 nm and 230 nm.

Such a filtering or blocking can e.g. be achieved by designing arc tube 1 or outer tube 8 to act as a blocking filter. Such Filters can e.g. be formed by layers deposited on the tubes, or by intrinsic properties of the tube material.

In an advantageous embodiment, the tube material for one or both tubes 1, 8 comprises synthetic quartz glass with embedded nanoparticles of electrically conducting or dielectric materials. The theoretical basics of such filter devices (Mie-filter) are described e.g. in M. Born, E. Wolf, “Principles of Optics”, Pergamon Press, Oxford 1980, 633-664.

The danger of a bursting of arc tube 1 increases during the life time of a high-pressure mercury lamp, in particular due to recrystallization of the quartz glass material at high temperatures. A certain base probability for a bursting exists at any time. However, mercury vapor leaking into the chamber would contaminate the same thoroughly.

In the embodiment of FIG. 1, the mercury remains contained within outer tube 8. The risk of outer tube 8 bursting at the same time is smaller by orders of magnitude since it does not experience recrystallization during operation and can have thicker walls.

The lamp body consisting of the components 1 to 9 and 13 has cylindrical shape and is provided with sockets 10 at both ends. Each socket 10 consists of metal, e.g. stainless steel or light metal. They are joined in air-tight (vacuum-tight) manner with the lamp body and in particular also with the electric insulator 11 of the electric conductors, e.g. by means of a cement. An air-tight seal prevents an oxidation of the lead-throughs 3 at the high temperatures in the lamp. Furthermore, the air tight seal prevents an access of water vapor to the lead throughs during the biological preparation while the lamp is switched off.

The sockets 10 are connected to heat sinks 12, e.g. of light metal, via the contact surfaces 16. The heat sinks allow to operate the lamp at elevated environmental temperatures of e.g. 440 K. The surfaces 16 are covered with heat conducting paste. The heat sinks 12 provide a high heat conductance between the lamp and its environment in order to obtain a large heat flow even if the temperature difference between the lamp and its environment is comparatively small.

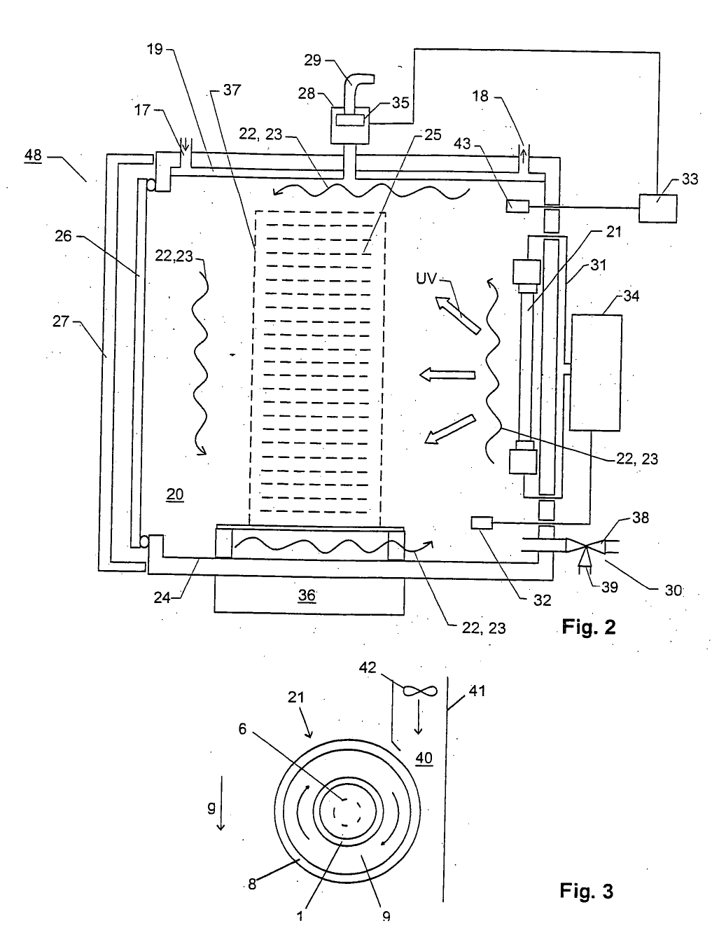

FIG. 2 shows a schematic cross section through a climate controlled cabinet according to the present invention. It comprises a climate control 36, which controls the temperature and, if desired, the atmosphere in the chamber 20, and, in particular, is able to establish a well defined temperature and (optionally) humidity.

Storage locations for receiving laboratory goods or probes are provided in chamber 20. Advantageously, these are formed by one or more storage racks 37, which comprise a plurality of lateral ledges 25 to define a plurality of storage locations arranged above each other.

The storage racks 37 can be stationary or they can be mounted to a rotating carousel. Furthermore, the climate controlled cabinet can further be equipped with a transport unit for automatic access to the goods/probes and/or with a shaker for shaking the goods/probes. Corresponding devices are e.g. shown in WO 02/059253.

FIG. 2 shows the high-pressure mercury lamp 21 in chamber 20. Advantageously, a high-pressure mercury lamp as shown in FIG. 1 is used. FIG. 2 also shows the flows of air 22, ozone 23 and UV-radiation (UV). The interior walls of chamber 20 advantageously have surfaces that reflect UV-radiation, e.g. of stainless steel.

The goods/samples and/or storage racks 37 can e.g. be brought into and removed from chamber 20 through front- or user-doors 26, 27. Two doors are provided. An inner front door 26 consists partially of UV-absorbing glass that is transparent for visible light and an outer front door of non-transparent, radiation absorbing material, such as steel. This arrangement allows to temporarily open the outer door even during operation of the high-pressure mercury lamp for inspecting chamber 20. In normal sterilization operation, however, outer front door 27 should remain closed for safety reasons.

An electronic safety circuit is provided for switching off high-pressure mercury lamp 21 when outer front door 27 is opened for a time span exceeding a safety margin. Inner front door 26 remains mechanically locked at all times while high-pressure mercury lamp 21 is in operation and, when the lamp is switched off, remains locked during an additional safety period.

As a further safety measure preventing ozone from leaking into the environment, a gas removal device 28 is provided. Gas removal device 28 comprises a low power pump that keeps chamber 20 during decontamination under slight underpressure to prevent an uncontrolled leakage of gas through possible leaks. The underpressure is controlled by means of a pressure sensor 43 and a pressure control loop 33 controlling the operation of the pump. A catalyzer 35 is arranged in the exit air duct 29 of the air removal device 28. Catalyzer 35 converts ozone to normal oxygen (O₂).

When high-pressure mercury lamp 21 is switched off, a valve 30, e.g. a three-way-valve, is operated to open a gas inlet 39. Valve 30 remains open during a safety period for allowing a quicker flushing of chamber 20 through gas removal device 28. Inner front door 26 can only be opened after expiry of the safety period.

Gas inlet 39 can also be used to feed oxygen to the chamber during decontamination. By increasing the oxygen amount, a larger ozone concentration can be achieved.

During decontamination, the temperature in chamber 20 is controlled by varying the electrical power fed to high-pressure mercury lamp 21. For this purpose, a temperature sensor 32 is arranged in chamber 20, the signal of which is fed to a control loop in lamp driver 34. The control loop controls the power fed through the feeds 31 to high-pressure mercury lamp 21 in such a manner that the temperature in chamber 20 remains within a given interval.

Theoretically, a temperature as high as possible, e.g. up to 440 K, is desirable in chamber 20 during decontamination. However, this temperature may not be allowable if further, temperature sensitive components (not shown in FIG. 2) are present in chamber 20. In that case the climate controlled cabinet is adjusted such that a suitable temperature range (or limit) is maintained during decontamination. As it is easily understood, the primary agents for decontamination will be UV-radiation and ozone in case that the given temperature limit is low. It must be noted, though, that it is possible to reach higher ozone levels at lower temperatures because ozone starts to decay at elevated temperatures.

In order to achieve a high UV radiation and ozone level even in the presence of a low temperature limit, upper wall 24 of chamber 20 is a heat sink wall 19 cooled by a cooling fluid. Heat sink wall 19 has a cavity for circulating the cooling fluid, such as air or water, from an inlet 17 to an outlet 18. By suitable adjustment of the fluid flow, a certain amount of heat can be carried off. A fine regulation of the temperature within chamber 20 can then e.g. be taken over by the control loop in lamp driver 34. Alternatively, the desired temperature interval in chamber 20 can be maintained even at constant lamp current if the flow of the cooling fluid through heat sink wall 19 is controlled by a suitable control loop. The fluid can, in its turn, e.g. be cooled by a heat pump.

FIG. 2 shows an embodiment of the invention where the high-pressure mercury lamp is mounted vertically, i.e. with vertical longitudinal axis 5. This orientation has certain advantages for the lamp itself as well as for the decontamination procedure.

Arc tube 1 of the lamp is subjected to very high temperatures during operation of the lamp. At these temperatures, the walls of arc tube 1 are somewhat softened, which can lead to a sagging if the lamp is arranged horizontally.

Within arc tube 1, an advantageous thermal convection of gas filling 6 builds up in known manner. A similar process is observed in the filling gas of gap 9. Hence, thermally stable conditions for a heat transport are created.

Similar physical processes take place in chamber 20. An upwards directed flow of air is generated along high-pressure mercury lamp 21, which air is heated in particular by outer tube 8 and the heat sinks 12.

The vertically mounted high-pressure mercury lamp induces a homogeneous distribution of the hot air as well as of the ozone generated close to the lamp. Even if the UV light is not subject to thermal convection, a central, vertical position is in most practical applications the most favorable one.

If, e.g. in the presence of special items within chamber 20, a horizontal arrangement of high-pressure mercury lamp 21 becomes necessary, a mechanical gas circulation pump should be arranged in chamber 20, namely in such a way that high-pressure mercury lamp 21 is cooled asymmetrically in respect to its longitudinal axis.

This thought is illustrated in FIG. 3, which shows a sectional view of the high-pressure mercury lamp of FIG. 1 in plane S. The figure further shows an air duct 40 with a forced air flow 41 generated by gas circulation pump 42. Arrow g shows the vertical down-direction.

In this case it is essential that only one of the vertical outer surfaces of outer tube 8 is cooled by the air flow. In this case, a circular thermal convention around arc tube 1 is formed in gap 9. This convection leads to stable thermal conditions in arc tube 1.

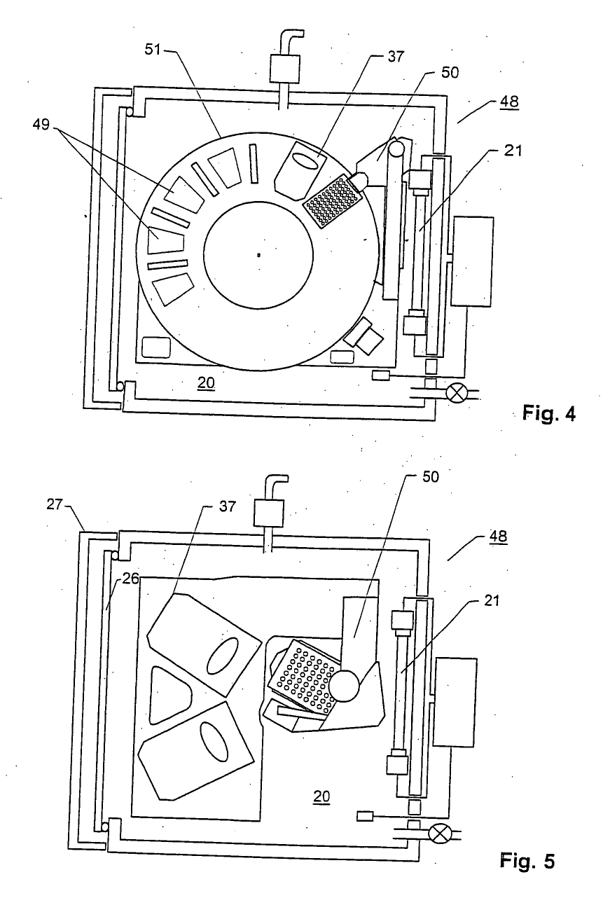

FIG. 4 shows a horizontal cross section through a climate controlled cabinet 48 with horizontally arranged high-pressure mercury lamp 21. In this embodiment, the storage racks 37 (only one of which is shown in FIG. 4) are arranged on a rotatable carousel and can be accessed by an automatic transport unit 50. Openings 49 in carousel 51 reduce the formation of shadows below the carousel and encourage a homogeneous distribution of UV radiation.

Using a high-pressure mercury lamp 21 within this type of chamber 20 is particularly advantageous in automated climate controlled cabinets 48, e.g. with a transport. unit 50 and/or carousel 51. In conventional climate controlled cabinets, these components have to be removed from chamber 20 for decontamination because conventional in-situ hot-air sterilization is unable to decontaminate a broad range of germs, in particular because the standard temperature of 440 K for hot air decontamination cannot be reached.

FIG. 5 also shows a horizontal section through a climate controlled cabinet with horizontally arranged high-pressure mercury lamp 21. In this embodiment, exactly two storage racks 37 are arranged with V-shaped footprints are provided. Again, the climate controlled cabinet comprises a transport unit 50, which is able to automatically access the items in the storage racks 37. Again, the climate controlled cabinet comprises a transport unit 50 for automatically accessing the goods/samples in the storage racks 37. Using a high-pressure mercury lamp in such an automated climate controlled cabinets is advantageous for the same reasons as mentioned in context with FIG. 4.

If parts in the cabinet are movable (such as the carousel of FIG. 4), they can be moved automatically during decontamination in order to increase the homogeneity of the UV-illumination.

1. A cabinet comprising
a chamber for a controlled storage or preparation of biological samples or goods and
at least one high-pressure mercury lamp located to sterilize said chamber prior to receiving said goods.

2. The cabinet of claim 1 wherein said high-pressure mercury lamp comprises
an arc tube and
an outer tube surrounding said arc tube for containing mercury in case of a failure of said arc tube.

3. The cabinet of claim 1 wherein said high-pressure mercury lamp comprises a rod member extending between a collar and said arc tube for holding said arc tube, wherein said outer tube is fused in gas tight manner to said rod member via said collar.

4. The cabinet of claim 3 comprising at least one electrical lead-through extending through said rod member.

5. The cabinet of claim 3 wherein said rod member is of glass or quartz glass.

6. The cabinet of claim 3 wherein a gap between said outer tube and said arc tube is filled with an oxygen-free gas, in particular nitrogen.

7. The cabinet of claim 1 wherein said high-pressure mercury lamp comprises at least one gas tight socket and an electric conductor leading through said socket, wherein said electric conductor is connected to said socket in gas tight manner.

8. The cabinet of claim 3 wherein said high-pressure mercury lamp comprises
at least one gas tight socket and
heat conducting paste arranged in a gap between said collar and said socket.

9. The cabinet of claim 1 comprising at least one heat sink connected to said high-pressure mercury lamp.

10. The cabinet of claim 9 wherein said heat sink is in thermal contact with a socket of said lamp with heat conducting paste arranged between said socket and said heat sink.

11. The cabinet of claim 2 wherein said high-pressure mercury lamp is adapted not to exceed a temperature of 700 K at an outer surface of said outer tube.

12. The cabinet of claim 1 wherein said high-pressure mercury lamp is located in or at said chamber to emit UV-light into said chamber directly or via reflectors.

13. The cabinet of claim 12 wherein interior surfaces of said chamber are reflective for UV-light.

14. The cabinet of claim 1 wherein said chamber is closeable in gas tight manner.

15. The cabinet of claim 1 having an inner door that is at UV-absorbing but at least partially transparent for visible light, and an outer, non- transparent door.

16. The cabinet of claim 1 further comprising a gas removal device for generating an underpressure in said chamber.

17. The cabinet of claim 16 comprising a catalyzer for decomposing ozone in gas removed by said gas removal device.

18. The cabinet of claim 17 further comprising a gas inlet and a valve for closing or opening said gas inlet.

19. The cabinet of claim 18 being adapted to decompose and flush said ozone in said chamber after switching off said high-pressure mercury lamp and before opening a door of said cabinet.

20. The cabinet of claim 18 adapted to increase an ozone concentration in said chamber by leading oxygen through said gas inlet into said chamber while maintaining an underpressure in said chamber by means of said gas removal device.

21. The cabinet of claim 1 comprising movable parts in said chamber, wherein said cabinet is adapted to move said movable parts for increasing a homogeneity of a UV-illumination in said chamber.

22. The cabinet of claim 1 wherein said high-pressure mercury lamp has a vertically aligned elongate axis.

23. The cabinet of claim 1 wherein said high-pressure mercury lamp ha-s a horizontally aligned elongate axis and is cooled asymmetrically.

24. The cabinet of claim 23 further comprising an air circulator for blowing air against only one vertical surface of said high-pressure mercury lamp, thereby generating a thermal convention within said high-pressure mercury lamp.

25. The cabinet of claim 1 wherein said high-pressure mercury lamp is arranged opposite to a user-operable door of said cabinet.

26. The cabinet. of claim 1 wherein said high-pressure mercury lamp is adapted to emit light with a wavelength of less than 250 nm, in particular less than 230 nm, into said chamber.

27. The cabinet of claim 1 comprising a control unit for controlling a temperature in said chamber by varying a power fed to said high-pressure mercury lamp.

28. The cabinet of claim 1 comprising a heat sink wall cooled by a cooling fluid for cooling said chamber while operating said high-pressure mercury lamp.

29. The cabinet of claim 1 comprising a climate control for controlling a climate in said chamber.

30. The cabinet of claim 1 wherein said high-pressure mercury lamp comprises an outer tube having a better optical transmission for radiation between 180 nm and 230 nm than for radiation between 230 nm and 280 nm.

31. The cabinet of claim 30 wherein said outer tube comprises quartz glass and nanoparticles suspended in said quartz glass.

32. A method for sterilizing a chamber of a cabinet for storing biological samples or goods comprising the step of sending UV-light from a high-pressure mercury lamp into said chamber while, at the same time, heating said chamber with heat from said high-pressure mercury lamp and generating ozone in said chamber with said UV-light.

33. A high-pressure or low-pressure mercury lamp comprising at least one optical filter having a better optical transmission for radiation between 180 nm and 230 nm than for radiation between 230 nm and 280 nm.

34. The high-pressure or low-pressure mercury lamp of claim 33 having at least one quartz glass tube comprising nanoparticles acting as said. optical filter.

35. A method for sterilizing a chamber of a cabinet for storing biological samples or goods comprising the steps of
cycling air in said chamber and, simultaneously,
sending UV-light from a high-pressure mercury lamp into said chamber, thereby generating a level of ozone lethal for germs.
